(19) United States
(12) Patent Application Publication
(10) Pub. No.: US 2016/0257672 A1
(43) Pub. Date: Sep. 8, 2016

(54) FUNCTIONALIZED FURAN-2-SULFONAMIDES EXHIBITING ENDOTHELIAL LIPASE INHIBITION

(71) Applicants: TEMPLE UNIVERSITY-OF THE COMMONWEALTH SYSTEM OF HIGHER EDUCATION, Philadelphia, PA (US); SHIFA BIOMEDICAL CORPORATION, Malvern, PA (US)

(72) Inventors: Magid Abou-Gharbia, Exton, PA (US); Wayne E. Childers, New Hope, PA (US); Rogelio Martinez, Trenton, NJ (US); Victor P. Ghidu, Philadelphia, PA (US); Harold Meyers, Weston, MA (US); Shaker A. Mousa, Wynantskill, NY (US); Nabil Elshourbagy, West Chester, PA (US)

(21) Appl. No.: 15/028,164

(22) PCT Filed: Oct. 6, 2014
(86) PCT No.: PCT/US14/59275

§ 371 Date: Apr. 8, 2016

Related U.S. Application Data
(60) Provisional application No. 61/888,144, filed on Oct. 8, 2013.

(51) Int. Cl. C07D 405/12; C07D 487/08; C07D 471/08

(52) U.S. Cl. CPC C07D 405/12; C07D 471/08; C07D 487/08

(57) ABSTRACT

The present invention relates to pharmaceutical compositions comprising furan-2-sulfonamide derivatives. The present invention further relates to methods of treatment of diseases or conditions associated with endothelial lipase activity, including coronary artery disease and low HDL-C.

### CROSS-REFERENCE TO RELATED APPLICATIONS

The present application claims priority to U.S. Provisional Patent Application No. 61/888,144, filed Oct. 8, 2013, which is herein incorporated by reference in its entirety.

### STATEMENT REGARDING FEDERALLY SPONSORED RESEARCH OR DEVELOPMENT

This invention was made with government support under Grant No. R44HL097438 awarded by the National Heart, Lung, and Blood Institute (NHLBI). The government has certain rights in the invention.

### BACKGROUND OF THE INVENTION

Lipid disorders are the major contributor of premature coronary artery disease (CAD). Intervention with drugs to reduce cholesterol has proven to decrease the risk of subsequent cardiovascular events, including morbidity and mortality. Based on a wealth of clinical data, it is widely agreed that patients with CAD should be treated with lipid-lowering drugs to reduce the risk of subsequent events. Although there have been considerable advancements in low density lipoprotein-cholesterol (LDL-C) lowering therapies in the past 20 years, a significant number of patients are unresponsive and remain at high cardiovascular risk. To address this concern, attention is now shifting toward strategies for targeting high density lipoprotein-cholesterol (HDL-C) as adjunctive therapy to prevent and treat cardiovascular disease. HDL may exert several potentially important anti-atherosclerotic, anti-inflammatory and endothelial-protective effects. Increasing evidence has emphasized that the risk factor associated with low levels of HDL-C is independent of that of high LDL-C(Petremand et al., 2008, Current Opinion in Lipidology 19: 95-97). Recent epidemiological data confirmed that patients with a low HDL-C level are at high risk of premature cardiovascular disease no matter how low the LDL-C level (Foody, 2006, Preventative Cardiology, Foody, ed., Humana Press). These and other patients may dramatically benefit from an aggressive treatment of low HDL-C levels. Currently marketed drugs for raising HDL-C are not very effective and have major side effects.

One possible method for developing agents that selectively raise HDL-C levels is to target the HDL-C degradation pathway, which is primarily mediated by endothelial lipase (EL). EL controls the degradation of HDL-C, and thereby contributes to HDL-C homeostasis. EL is a member of the triglyceride lipase gene family that includes lipoprotein lipase (LPL) and hepatic lipase (HL), but unlike LPL and HL, EL is synthesized by endothelial cells and functions at the site where it is synthesized. Furthermore, the tissue distribution for EL is different from that of LPL and HL, and has greater specificity than HL for phospholipids and for HDL-C. Over-expression of EL in mice results in reduction in HDL-C and Apo-A1 due to increased catabolism (Ma et. al., 2003 Proc. Nat'l. Acad. Sci. USA 100: 2748-2753), and loss of function of EL in double knockout and heterozygous mice results in a significant increase in HDL-C levels (Jin et al., 2003 J Clin Invest 111:357-362). In addition, studies in humans have suggested association of EL gene variants with high HDL-C levels and indicate that plasma EL concentration is inversely proportional to HDL-C levels (deLemos et. al., 2002 Circulation 106:1321-1326; Edmondson et. al., 2009, J Clin Invest 119:1042-1050). Therefore, EL is an attractive target for the development of drugs to raise HDL-C.

Thus, there is a need in the art for new therapeutic agents capable of raising HDL-C levels in patients with low HDL-C levels. There is also a clear and present need for medications capable of treating diseases that involve aberrant endothelial lipase activity. The present invention addresses these unmet needs in the art.

### SUMMARY OF THE INVENTION

The present invention is directed toward novel functionalized furan-2-sulfonamides, compounds of formula (I),

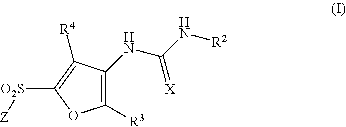

including hydrates, solvates, enantiomers, diastereomers, pharmaceutically acceptable salts, prodrugs and complexes thereof, wherein:
A is selected from the group consisting of CR^{1a}R^{1b}, sulfur, oxygen, and NR⁷;
X is selected from the group consisting of oxygen, sulfur, and NH;

### Z is

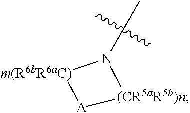

R^{1a} and R^{1b} are each independently selected from a group consisting of optionally substituted C₁-C₆ alkyl, optionally substituted C₃-C₈ cycloalkyl, optionally substituted aryl, optionally substituted arylalkyl, and optionally substituted heteroaryl;
R^{1a} and R^{1b} are taken together with the atoms to which they are bound to form a ring containing 3 to 6 atoms;
R² is selected from a group consisting of optionally substituted C₃-C₇ cycloalkyl, optionally substituted aryl, optionally substituted heteroaryl, optionally substituted biphenyl,

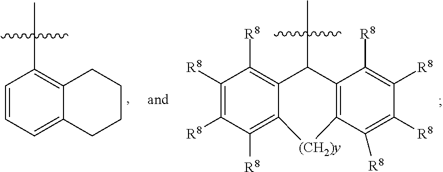

R³ is selected from the group consisting of hydrogen, C₁-C₆ alkyl, and C₃-C₇ cycloalkyl;
R⁴ is selected from the group consisting of hydrogen, C₁-C₆ alkyl, and C₃-C₇ cycloalkyl;
R^{5a} and R^{5b} are at each occurrence independently selected from the group consisting of hydrogen, optionally substituted C₁-C₆ alkyl, and optionally substituted C₃-C₇ cycloalkyl;
R^{5a} and R^{5b} are taken together with the atoms to which they are bound to form a ring containing 3 to 6 atoms;
R^{6a} and R^{6b} are at each occurrence independently selected from the group consisting of hydrogen, optionally substituted C₁-C₆ alkyl, and optionally substituted C₃-C₇ cycloalkyl;
R^{6a} and R^{6b} are taken together with the atoms to which they are bound to form a ring containing 3 to 6 atoms;
R^{5a} and R^{6b} are taken together with the atoms to which they are bound to form a ring containing 3 to 6 atoms;
R⁷ is selected from the group consisting of hydrogen, optionally substituted C₁-C₆ alkyl, and optionally substituted C₃-C₇ cycloalkyl;
R⁸ at each occurrence is independently selected from a group consisting of hydrogen, halogen, cyano, optionally substituted C₁-C₆ alkyl, optionally substituted C₁-C₆ alkoxy, optionally substituted C₃-C₈ cycloalkyl, OH, NH₂, NH(C₁-C₆ alkyl), N(C₁-C₆ alkyl)₂, NO₂, C₁-C₃ haloalkyl, C₁-C₃ haloalkoxy, SH, S C₁-C₆alkyl, and 3-10 membered cycloheteroalkyl containing 1 to 4 heteroatoms selected from N, O and S;
m=1, 2, 3, or 4;
n=1, 2, 3, or 4;
y=0, 1, or 2;
with the proviso that the compound of the present invention excludes the compound of the formula (II):

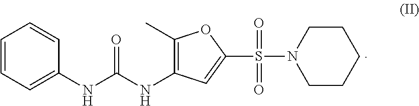 [SMILES: Cc1oc(S(=O)(=O)N2CCCCC2)cc1NC(=O)Nc1ccccc1]

The present invention further relates to compositions comprising an effective amount of one or more compounds according to the present invention and an excipient.

The present invention also relates to a method for treating or preventing diseases that involve low HDL-C levels, including, for example, coronary artery disease, said method comprising administering to a subject an effective amount of a compound or composition according to the present invention.

The present invention yet further relates to a method for treating or preventing diseases that involve low HDL-C levels, including, for example, coronary artery disease, wherein said method comprises administering to a subject a composition comprising an effective amount of one or more compounds according to the present invention and an excipient.

The present invention also relates to a method for treating or preventing disease or conditions associated with coronary artery disease, and diseases that involve low HDL-C levels. Said methods comprise administering to a subject an effective amount of a compound or composition according to the present invention.

The present invention yet further relates to a method for treating or preventing disease or conditions associated with coronary artery disease, and diseases that involve low HDL-C levels, wherein said method comprises administering to a subject a composition comprising an effective amount of one or more compounds according to the present invention and an excipient.

The present invention also relates to a method for treating or preventing disease or conditions associated with aberrant endothelial lipase activity. Said methods comprise administering to a subject an effective amount of a compound or composition according to the present invention.

The present invention yet further relates to a method for treating or preventing disease or conditions associated with aberrant endothelial lipase activity, wherein said method comprises administering to a subject a composition comprising an effective amount of one or more compounds according to the present invention and an excipient.

The present invention further relates to a process for preparing the endothelial lipase inhibitors of the present invention.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference; the citation of any document is not to be construed as an admission that it is prior art with respect to the present invention.

### DETAILED DESCRIPTION

It is to be understood that the figures and descriptions of the present invention have been simplified to illustrate elements that are relevant for a clear understanding of the present invention, while eliminating, for the purpose of clarity, many other elements found in organic and medicinal chemistry. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the description, where an element or component is said to be included in and/or selected from a list of recited elements or components, it should be understood that the element or component can be any one of the recited elements or components and can be selected from a group consisting of two or more of the recited elements or components.

It should be understood that the order of steps or order for performing certain actions is immaterial so long as the present teachings remain operable. Moreover, two or more steps or actions can be conducted simultaneously.

Unless defined otherwise, all technical and scientific terms used herein have the same meaning as commonly understood by one of ordinary skill in the art to which this invention belongs. Although any methods, materials and components similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate. As used herein, the term “halogen” shall mean chlorine, bromine, fluorine and iodine.

As used herein, unless otherwise noted, “alkyl” and/or “aliphatic” whether used alone or as part of a substituent group refers to straight and branched carbon chains having 1 to 20 carbon atoms or any number within this range, for example 1 to 6 carbon atoms or 1 to 4 carbon atoms. Designated numbers of carbon atoms (e.g. C₁-C₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁-C₆alkyl)₂amino, the alkyl groups may be the same or different.

As used herein, the terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups can be optionally substituted. Nonlimiting examples of alkenyl groups include ethenyl, 3-propenyl, 1-propenyl (also 2-methylethenyl), isopropenyl (also 2-methylethen-2-yl), buten-4-yl, and the like. Nonlimiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Nonlimiting examples of alkynyl groups include ethynyl, prop-2-ynyl (also propargyl), propyn-1-yl, and 2-methyl-hex-4-yn-1-yl. Nonlimiting examples of substituted alkynyl groups include 5-hydroxy-5-methylhex-3-ynyl, 6-hydroxy-6-methylhept-3-yn-2-yl, 5-hydroxy-5-ethylhept-3-ynyl, and the like.

As used herein, “cycloalkyl,” whether used alone or as part of another group, refers to a non-aromatic carbon-containing ring including cyclized alkyl, alkenyl, and alkynyl groups, e.g., having from 3 to 14 ring carbon atoms, preferably from 3 to 7 or 3 to 6 ring carbon atoms, or even 3 to 4 ring carbon atoms, and optionally containing one or more (e.g., 1, 2, or 3) double or triple bond. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Cycloalkyl rings can be optionally substituted. Nonlimiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

“Haloalkyl” is intended to include both branched and straight-chain saturated aliphatic hydrocarbon groups having the specified number of carbon atoms, substituted with 1 or more halogen. Haloalkyl groups include perhaloalkyl groups, wherein all hydrogens of an alkyl group have been replaced with halogens (e.g., —CF₃, —CF₂CF₃). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

The term “alkoxy” refers to the group —O-alkyl, wherein the alkyl group is as defined above. Alkoxy groups optionally may be substituted. The term C₃-C₆ cyclic alkoxy refers to a ring containing 3 to 6 carbon atoms and at least one oxygen atom (e.g., tetrahydrofuran, tetrahydro-2H-pyran). C₃-C₆ cyclic alkoxy groups optionally may be substituted.

The term “aryl,” wherein used alone or as part of another group, is defined herein as a an unsaturated, aromatic monocyclic ring of 6 carbon members or to an unsaturated, aromatic polycyclic ring of from 10 to 14 carbon members. Aryl rings can be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

The term “arylalkyl” or “aralkyl” refers to the group -alkyl-aryl, where the alkyl and aryl groups are as defined herein. Aralkyl groups of the present invention are optionally substituted. Examples of arylalkyl groups include, for example, benzyl, 1-phenylethyl, 2-phenylethyl, 3-phenylpropyl, 2-phenylpropyl, fluorenylmethyl and the like.

The terms “heterocyclic” and/or “heterocycle” and/or “heterocylyl,” whether used alone or as part of another group, are defined herein as one or more ring having from 3 to 20 atoms wherein at least one atom in at least one ring is a heteroatom selected from nitrogen (N), oxygen (O), or sulfur (S), and wherein further the ring that includes the heteroatom is non-aromatic. In heterocycle groups that include 2 or more fused rings, the non-heteroatom bearing ring may be aryl (e.g., indolinyl, tetrahydroquinolinyl, chromanyl). Exemplary heterocycle groups have from 3 to 14 ring atoms of which from 1 to 5 are heteroatoms independently selected from nitrogen (N), oxygen (O), or sulfur (S). One or more N or S atoms in a heterocycle group can be oxidized. Heterocycle groups can be optionally substituted.

Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl.

The term “heteroaryl,” whether used alone or as part of another group, is defined herein as one or more rings having from 5 to 20 atoms wherein at least one atom in at least one ring is a heteroatom chosen from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl (e.g., benzofuranyl, benzothiophenyl, indolyl). Exemplary heteroaryl groups have from 5 to 14 ring atoms and contain from 1 to 5 ring heteroatoms independently selected from nitrogen (N), oxygen (O), or sulfur (S). One or more N or S atoms in a heteroaryl group can be oxidized. Heteroaryl groups can be substituted. Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, and isoquinolinyl.

One non-limiting example of a heteroaryl group as described above is C₁-C₅ heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., R² and R³ taken together with the nitrogen (N) to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

For the purposed of the present invention fused ring units, as well as spirocyclic rings, bicyclic rings and the like, which comprise a single heteroatom will be considered to belong to the cyclic family corresponding to the heteroatom containing ring. For example, 1,2,3,4-tetrahydroquinoline having the formula:

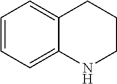

is, for the purposes of the present invention, considered a heterocyclic unit. 6,7-Dihydro-5H-cyclopentapyrimidine having the formula:

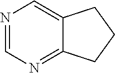

is, for the purposes of the present invention, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

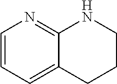

is, for the purposes of the present invention, considered a heteroaryl unit.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is used throughout the specification. The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, can be optionally substituted. Optionally substituted groups will be so indicated.

The following are non-limiting examples of substituents which can substitute for hydrogen atoms on a moiety: halogen (chlorine (Cl), bromine (Br), fluorine (F) and iodine(I)), —CN, —NO₂, oxo (═O), —OR⁹, —SR⁹, —N(R⁹)₂, —NR⁹C(O)R⁹, —SO₂R⁹, —SO₂OR⁹, —SO₂N(R⁹)₂, —C(O)R⁹, —C(O)OR⁹, —C(O)N(R⁹)₂, C₁-C₆ alkyl, C₁-C₆haloalkyl, C₁-C₆alkoxy, C₂-C₈alkenyl, C₂-C₈ alkynyl, C₃₋₁₄ cycloalkyl, aryl, heterocycle, or heteroaryl, wherein each of the alkyl, haloalkyl, alkenyl, alkynyl, alkoxy, cycloalkyl, aryl, heterocycle, and heteroaryl groups is optionally substituted with 1-10 (e.g., 1-6 or 1-4) groups selected independently from halogen, —CN, —NO₂, oxo, and R⁹; wherein R⁹, at each occurrence, independently is hydrogen, —OR¹⁰, —SR¹⁰, —C(O)R¹⁰, C(O)OR¹⁰, —) C(O)N(R¹⁰)₂, —SO₂R¹⁰, —S(O)₂OR¹⁰, —N(R¹⁰)₂, —NR¹⁰C(O)R¹⁰, C₁-C₆ alkyl, C₁-C₆ haloalkyl, C₂-C₈ alkenyl, C₂-C₈ alkynyl, cycloalkyl (e.g., C₃₋₆ cycloalkyl), aryl, heterocycle, or heteroaryl, or two R⁹ units taken together with the atom(s) to which they are bound form an optionally substituted carbocycle or heterocycle wherein said carbocycle or heterocycle has 3 to 7 ring atoms; wherein R¹⁰, at each occurrence, independently is hydrogen, C₁-C₆ alkyl, C₁-C₆ haloalkyl, C₂-C₈ alkenyl, C₂-C₈ alkynyl, cycloalkyl (e.g., C₃₋₆ cycloalkyl), aryl, heterocycle, or heteroaryl, or two R¹⁰ units taken together with the atom(s) to which they are bound form an optionally substituted carbocycle or heterocycle wherein said carbocycle or heterocycle preferably has 3 to 7 ring atoms.

In various embodiments, the substituents can be selected from:
i) —OR¹¹; for example, —OH, —OCH₃, —OCH₂CH₃, —OCH₂CH₂CH₃;
ii) —C(O)R¹¹; for example, —COCH₃, —COCH₂CH₃, —COCH₂CH₂CH₃;
iii) —C(O)OR¹¹; for example, —CO₂CH₃, —CO₂CH₂CH₃, —CO₂CH₂CH₂CH₃;
iv) —C(O)N(R¹¹)₂; for example, —CONH₂, —CONHCH₃, —CON(CH₃)₂;
v) —N(R¹¹)₂; for example, —NH₂, —NHCH₃, —N(CH₃)₂, —NH(CH₂CH₃);
vi) halogen: —F, —Cl, —Br, and —I;
vii) —CHₑX_{g}; wherein X is halogen, m is from 0 to 2, e+g=3; for example, —CH₂F, —CHF₂, —CF₃, —CCl₃, or —CBr₃;
viii) —SO₂R¹¹; for example, —SO₂H; —SO₂CH₃; —SO₂C₆H₅;
ix) C₁-C₆ linear, branched, or cyclic alkyl;
x) Cyano
xi) Nitro;
xii) N(R¹¹)C(O)R¹¹;
xiii) Oxo (═O);
xiv) Heterocycle; and
xv) Heteroaryl.

wherein each R¹¹ is independently hydrogen, optionally substituted C₁-C₆ linear or branched alkyl (e.g., optionally substituted C₁-C₄ linear or branched alkyl), or optionally substituted C₃-C₆ cycloalkyl (e.g., optionally substituted C₃-C₄ cycloalkyl); or two R¹¹ units can be taken together to form a ring comprising 3-7 ring atoms. In certain aspects, each R¹¹ is independently hydrogen, C₁-C₆ linear or branched alkyl optionally substituted with halogen or C₃-C₆ cycloalkyl or C₃-C₆ cycloalkyl.

At various places in the present specification, substituents of compounds are disclosed in groups or in ranges. It is specifically intended that the description include each and every individual sub-combination of the members of such groups and ranges. For example, the term “C₁-C₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃—O₅, C₃-C₄, C₄-C₆, C₄-C₅, and C₅-C₆, alkyl.

For the purposes of the present invention the terms “compound,” “analog,” and “composition of matter” stand equally well for the endothelial lipase inhibitors described herein, including all enantiomeric forms, diastereomeric forms, salts, and the like, and the terms “compound,” “analog,” and “composition of matter” are used interchangeably throughout the present specification.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

When any variable occurs more than one time in any constituent or in any formula, its definition in each occurrence is independent of its definition at every other occurrence (e.g., in N(R¹⁰)₂, each R¹⁰ may be the same or different than the other). Combinations of substituents and/or variables are permissible only if such combinations result in stable compounds.

The terms “treat” and “treating” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that may be associated with the targeted or suspected disease or condition. These and other routine methods allow the clinician to select patients in need of therapy using the methods and compounds of the present invention.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

### DESCRIPTION

The present invention relates to compounds and methods useful as inhibitors of endothelial lipase, useful for the treatment of coronary artery disease and related conditions. The present invention further relates to a novel chemotype useful for the treatment of diseases that involve aberrant endothelial lipase activity.

The endothelial lipase inhibitors of the present invention can be used to treat or prevent diseases associated with low HDL-C levels, for example, coronary artery disease. The endothelial lipase inhibitors of the present invention can also be used to treat or prevent diseases associated with aberrant endothelial lipase activity. It has been discovered that patients with a low HDL-C level are at high risk of premature cardiovascular disease, and that inhibition of endothelial lipase causes increased HDL-C levels. Without wishing to be limited by theory, it is believed that the endothelial lipase inhibitors of the present invention can ameliorate, abate, or otherwise control, diseases associated with low HDL-C levels. It is further believed that the endothelial lipase inhibitors of the present invention can ameliorate, abate, or otherwise control, diseases associated with aberrant endothelial lipase activity.

### Compositions

The endothelial lipase inhibitors of the present invention are furan-2-sulfonamides, and include all enantiomeric and diastereomeric forms and pharmaceutically accepted salts thereof having the formula:

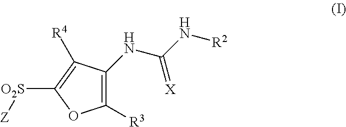

including hydrates, solvates, enantiomers, diastereomers, pharmaceutically acceptable salts, prodrugs and complexes thereof, wherein:
A is selected from the group consisting of CR^{1a}R^{1b}, Sulfur, Oxygen, and NR⁷;
X is selected from the group consisting of oxygen, sulfur, and NH;

### Z is

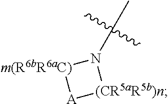

R^{1a} and R^{1b} are each independently selected from a group consisting of optionally substituted C₁-C₆ alkyl, optionally substituted C₃-C₈cycloalkyl, optionally substituted aryl, optionally substituted arylalkyl, and optionally substituted heteroaryl;
R^{1a} and R^{1b} are taken together with the atoms to which they are bound to form a ring containing 3 to 6 atoms;
R² is selected from a group consisting of optionally substituted C₃-C₇ cycloalkyl, optionally substituted aryl, optionally substituted heteroaryl, optionally substituted biphenyl,

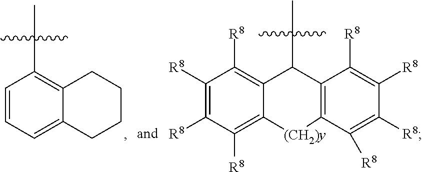

R³ is selected from the group consisting of hydrogen, C₁-C₆ alkyl, and C₃-C₇ cycloalkyl;
R⁴ is selected from the group consisting of hydrogen, C₁-C₆ alkyl, and C₃-C₇ cycloalkyl;
R^{5a} and R^{5b} are at each occurrence independently selected from the group consisting of hydrogen, optionally substituted C₁-C₆ alkyl, and optionally substituted C₃-C₇ cycloalkyl;
R^{5a} and R^{5b} are taken together with the atoms to which they are bound to form a ring containing 3 to 6 atoms;
R^{6a} and R^{6b} are at each occurrence independently selected from the group consisting of hydrogen, optionally substituted C₁-C₆ alkyl, and optionally substituted C₃-C₇ cycloalkyl;
R^{6a} and R^{6b} are taken together with the atoms to which they are bound to form a ring containing 3 to 6 atoms;
R^{5a} and R^{6b} are taken together with the atoms to which they are bound to form a ring containing 3 to 6 atoms;
R⁷ is selected from the group consisting of hydrogen, optionally substituted C₁-C₆ alkyl, and optionally substituted C₃-C₇ cycloalkyl;
R⁸ at each occurrence is independently selected from a group consisting of hydrogen, halogen, cyano, optionally substituted C₁-C₆ alkyl, optionally substituted C₁-C₆ alkoxy, optionally substituted C₃-C₈ cycloalkyl, OH, NH₂, NH(C₁-C₆ alkyl), N(C₁-C₆ alkyl)₂, NO₂, C₁-C₃ haloalkyl, C₁-C₃ haloalkoxy, SH, S (C₁-C₆ alkyl), and 3-10 membered cycloheteroalkyl containing 1 to 4 heteroatoms selected from N, O and S;
m=1, 2, 3, or 4;
n=1, 2, 3, or 4;
y=0, 1, or 2;
The compound of the present invention excludes the compound of the formula (II):

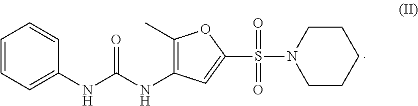 [SMILES: Cc1oc(S(=O)(=O)N2CCCCC2)cc1NC(=O)Nc1ccccc1]

The compounds of the present invention include compounds having formula (III):

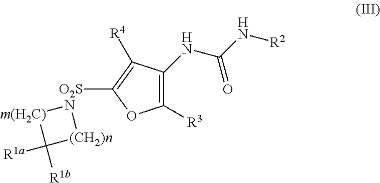

Including hydrates, solvates, pharmaceutically acceptable salts, prodrugs and complexes thereof.

The compounds of the present invention include compounds having formula (IV):

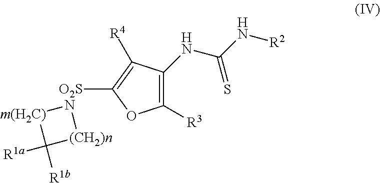

Including hydrates, solvates, pharmaceutically acceptable salts, prodrugs and complexes thereof.

The compounds of the present invention include compounds having formula (V):

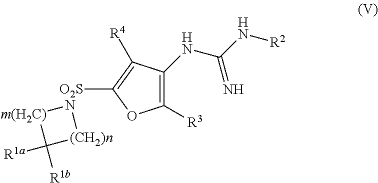

Including hydrates, solvates, pharmaceutically acceptable salts, prodrugs and complexes thereof.

The compounds of the present invention include compounds having formula (VI):

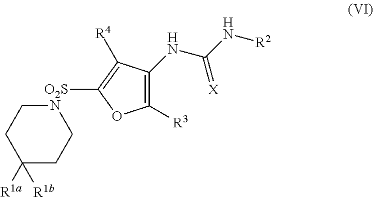

Including hydrates, solvates, pharmaceutically acceptable salts, prodrugs and complexes thereof.

The compounds of the present invention include compounds having formula (VII):

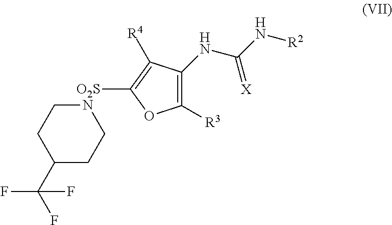

Including hydrates, solvates, pharmaceutically acceptable salts, prodrugs and complexes thereof.

In one embodiment of the present invention, Z is:

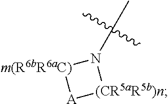

In one embodiment, A is CR^{1a}R^{1b}.

In one embodiment, A is sulfur.

In one embodiment, A is oxygen.

In one embodiment, A is NR⁷.

In one embodiment, X is oxygen.

In one embodiment, X is sulfur.

In one embodiment, X is NH.

In one embodiment, R^{1a} is an optionally substituted C₁-C₆ alkyl.

In one embodiment, R^{1a} is an optionally substituted C₃-C₈ cycloalkyl.

In one embodiment, R^{1a} is an optionally substituted aryl.

In one embodiment, R^{1a} is an optionally substituted arylalkyl.

In one embodiment, R^{1a} is an optionally substituted heteroaryl.

In one embodiment, R^{1b} is an optionally substituted C₁-C₆ alkyl.

In one embodiment, R^{1b} is an optionally substituted C₃-C₈ cycloalkyl.

In one embodiment, R^{1b} is an optionally substituted aryl.

In one embodiment, R^{1b} is an optionally substituted arylalkyl.

In one embodiment, R^{1b} is an optionally substituted heteroaryl.

In one embodiment, R^{1a} and R^{1b} are taken together with the atoms to which they are bound to form a ring containing 3 atoms.

In one embodiment, R^{1a} and R^{1b} are taken together with the atoms to which they are bound to form a ring containing 4 atoms.

In one embodiment, R^{1a} and R^{1b} are taken together with the atoms to which they are bound to form a ring containing 5 atoms.

In one embodiment, R^{1a} and R^{1b} are taken together with the atoms to which they are bound to form a ring containing 6 atoms.

In one embodiment, R² is optionally substituted C₃-C₇ cycloalkyl.

In one embodiment, R² is an optionally substituted aryl.

In one embodiment, R² is an optionally substituted heteroaryl.

In one embodiment, R² is an optionally substituted biphenyl.

In one embodiment, R² is

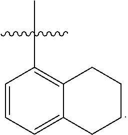 [SMILES: *c1cccc2c1CCCC2]

In one embodiment, R² is

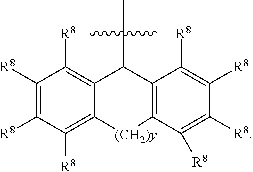

In one embodiment, R³ is hydrogen.

In one embodiment, R³ is a C₁-C₆ alkyl.

In one embodiment, R³ is a C₃-C₇ cycloalkyl.

In one embodiment, R⁴ is hydrogen.

In one embodiment, R⁴ is a C₁-C₆ alkyl.

In one embodiment, R⁴ is a C₃-C₇ cycloalkyl.

In one embodiment, R^{5a} is hydrogen.

In one embodiment, R^{5a} is an optionally substituted C₁-C₆ alkyl.

In one embodiment, R^{5a} is an optionally substituted C₃-C₇ cycloalkyl.

In one embodiment, R^{5b} is hydrogen.

In one embodiment, R^{5b} is an optionally substituted C₁-C₆ alkyl.

In one embodiment, R^{5b} is an optionally substituted C₃-C₇ cycloalkyl.

In one embodiment, R^{5a} and R^{5b} are taken together with the atoms to which they are bound to form a ring containing 3 atoms.

In one embodiment, R^{5a} and R^{5b} are taken together with the atoms to which they are bound to form a ring containing 4 atoms.

In one embodiment, R^{5a} and R^{5b} are taken together with the atoms to which they are bound to form a ring containing 5 atoms.

In one embodiment, R^{5a} and R^{5b} are taken together with the atoms to which they are bound to form a ring containing 6 atoms.

In one embodiment, R^{6a} is hydrogen.

In one embodiment, R^{6a} is an optionally substituted C₁-C₆ alkyl.

In one embodiment, R^{6a} is an optionally substituted C₃-C₇ cycloalkyl.

In one embodiment, R^{6b} is hydrogen.

In one embodiment, R^{6b} is an optionally substituted C₁-C₆ alkyl.

In one embodiment, R^{6b} is an optionally substituted C₃-C₇ cycloalkyl.

In one embodiment, R^{6a} and R^{6b} are taken together with the atoms to which they are bound to form a ring containing 3 atoms.

In one embodiment, R^{6a} and R^{6b} are taken together with the atoms to which they are bound to form a ring containing 4 atoms.

In one embodiment, R^{6a} and R^{6b} are taken together with the atoms to which they are bound to form a ring containing 5 atoms.

In one embodiment, R^{6a} and R^{6b} are taken together with the atoms to which they are bound to form a ring containing 6 atoms.

In one embodiment, R^{5a} and R^{6b} are taken together with the atoms to which they are bound to form a ring containing 3 atoms.

In one embodiment, R^{5a} and R^{6b} are taken together with the atoms to which they are bound to form a ring containing 4 atoms.

In one embodiment, R^{5a} and R^{6b} are taken together with the atoms to which they are bound to form a ring containing 5 atoms.

In one embodiment, R^{5a} and R^{6b} are taken together with the atoms to which they are bound to form a ring containing 6 atoms.

In one embodiment, R⁷ is hydrogen.

In one embodiment, R⁷ is an optionally substituted C₁-C₆ alkyl.

In one embodiment, R⁷ is an optionally substituted C₃-C₇ cycloalkyl.

In one embodiment, R⁸ is hydrogen.

In one embodiment, R⁸ is halogen.

In one embodiment, R⁸ is cyano.

In one embodiment, R⁸ is an optionally substituted C₁-C₆ alkyl.

In one embodiment, R⁸ is an optionally substituted C₁-C₆ alkoxy.

In one embodiment, R⁸ is an optionally substituted C₃-C₈ cycloalkyl.

In one embodiment, R⁸ is OH.

In one embodiment, R⁸ is NH₂.

In one embodiment, R⁸ is NH(C₁-C₆ alkyl).

In one embodiment, R⁸ is N(C₁-C₆ alkyl)₂.

In one embodiment, R⁸ is NO₂.

In one embodiment, R⁸ is a C₁-C₃ haloalkyl.

In one embodiment, R⁸ is a C₁-C₃ haloalkoxy.

In one embodiment, R⁸ is SH.

In one embodiment, R⁸ is S (C₁-C₆ alkyl).

In one embodiment, R⁸ is a 3-10 membered cycloheteroalkyl containing 1 to 4 heteroatoms selected from N, O and S.

In one embodiment, m is 1.

In one embodiment, m is 2.

In one embodiment, m is 3.

In one embodiment, m is 4.

In one embodiment, n is 1.

In one embodiment, n is 2.

In one embodiment, n is 3.

In one embodiment, n is 4.

In one embodiment, y is 0.

In one embodiment, y is 1.

In one embodiment, y is 2.

Exemplary embodiments of the present invention include a compound of Formula (I) or a pharmaceutically acceptable salt form thereof:

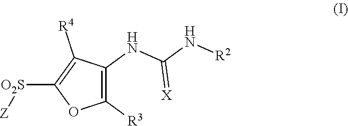

wherein non-limiting examples of Z, R², R³, R⁴, and X are defined herein below in Table 1.

TABLE 1

| Entry | Z | R² | R³ | R⁴ | X |
|---|---|---|---|---|---|
| 1 | 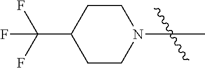 [SMILES: *N1CCC(C(F)(F)F)CC1] | Phenyl | CH₃ | H | O |
| 2 | 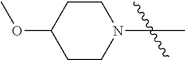 [SMILES: *N1CCC(OC)CC1] | Phenyl | CH₃ | H | O |
| 3 | 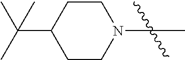 [SMILES: *N1CCC(C(C)(C)C)CC1] | Phenyl | CH₃ | H | O |
| 4 | 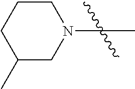 [SMILES: *N1CCCC(C)C1] | Phenyl | CH₃ | H | O |
| 5 | 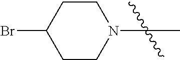 [SMILES: *N1CCC(Br)CC1] | Phenyl | CH₃ | H | O |
| 6 | 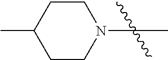 [SMILES: *N1CCC(C)CC1] | 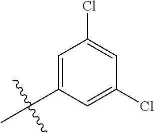 [SMILES: *c1cc(Cl)cc(Cl)c1] | CH₃ | H | O |
| 7 | 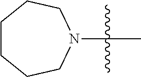 [SMILES: *N1CCCCCC1] | Phenyl | CH₃ | H | O |
| 8 | 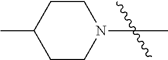 [SMILES: *N1CCC(C)CC1] | Phenyl | CH₃ | H | O |
| 9 | 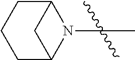 [SMILES: *N1C2CCCC1C2] | Phenyl | CH₃ | H | O |
| 10 | 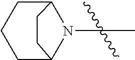 [SMILES: *N1C2CCCC1CC2] | Phenyl | CH₃ | H | O |
| 11 | 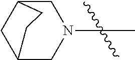 [SMILES: *N1CC2CCC(C2)C1] | Phenyl | CH₃ | H | O |
| 12 | 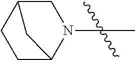 [SMILES: *N1CC2CCC1C2] | Phenyl | CH₃ | H | O |
| 13 | 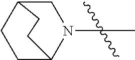 [SMILES: *N1CC2CCC1CC2] | Phenyl | CH₃ | H | O |
| 14 | 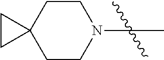 [SMILES: *N1CCC2(CC1)CC2] | Phenyl | CH₃ | H | O |
| 15 | 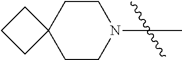 [SMILES: *N1CCC2(CCC2)CC1] | Phenyl | CH₃ | H | O |
| 16 | 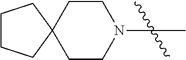 [SMILES: *N1CCC2(CCCC2)CC1] | Phenyl | CH₃ | H | O |
| 17 | 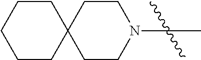 [SMILES: *N1CCC2(CCCCC2)CC1] | Phenyl | CH₃ | H | O |
| 18 | 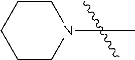 [SMILES: *N1CCCCC1] | 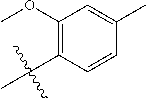 | CH₃ | H | O |
| 19 | 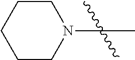 [SMILES: *N1CCCCC1] | 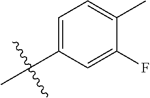 [SMILES: *c1ccc(C)c(F)c1] | CH₃ | H | O |
| 20 | 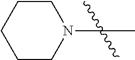 [SMILES: *N1CCCCC1] | 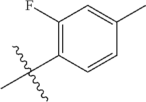 [SMILES: *c1ccc(C)cc1F] | CH₃ | H | O |
| 21 | 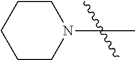 [SMILES: *N1CCCCC1] | 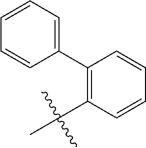 [SMILES: *c1ccccc1-c1ccccc1] | CH₃ | H | O |
| 22 | 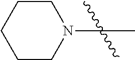 [SMILES: *N1CCCCC1] | 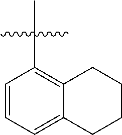 [SMILES: *c1cccc2c1CCCC2] | CH₃ | H | O |
| 23 | 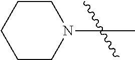 [SMILES: *N1CCCCC1] | 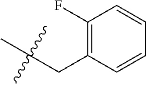 [SMILES: *Cc1ccccc1F] | CH₃ | H | O |
| 24 | 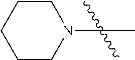 [SMILES: *N1CCCCC1] | 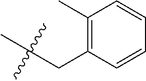 [SMILES: *Cc1ccccc1C] | CH₃ | H | O |
| 25 | 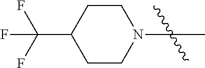 [SMILES: *N1CCC(C(F)(F)F)CC1] | Phenyl | CH₂CH₃ | H | O |
| 26 | 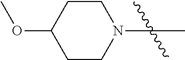 [SMILES: *N1CCC(OC)CC1] | Phenyl | CH₂CH₃ | H | O |
| 27 | 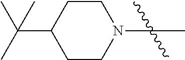 [SMILES: *N1CCC(C(C)(C)C)CC1] | Phenyl | CH₂CH₃ | H | O |
| 28 | 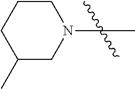 [SMILES: *N1CCCC(C)C1] | Phenyl | CH₂CH₃ | H | O |
| 29 | 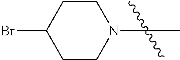 [SMILES: *N1CCC(Br)CC1] | Phenyl | CH₂CH₃ | H | O |
| 30 | 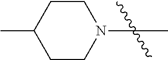 [SMILES: *N1CCC(C)CC1] | 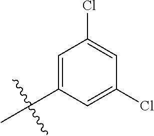 [SMILES: *c1cc(Cl)cc(Cl)c1] | CH₂CH₃ | H | O |
| 31 | 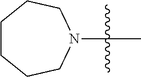 [SMILES: *N1CCCCCC1] | Phenyl | CH₂CH₃ | H | O |
| 32 | 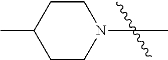 [SMILES: *N1CCC(C)CC1] | Phenyl | CH₂CH₃ | H | O |
| 33 | 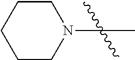 [SMILES: *N1CCCCC1] | 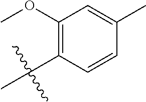 | CH₂CH₃ | H | O |
| 34 | 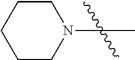 [SMILES: *N1CCCCC1] | 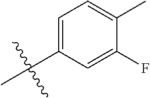 [SMILES: *c1ccc(C)c(F)c1] | CH₂CH₃ | H | O |
| 35 | 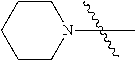 [SMILES: *N1CCCCC1] | 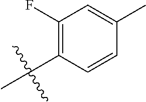 [SMILES: *c1ccc(C)cc1F] | CH₂CH₃ | H | O |
| 36 | 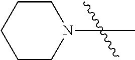 [SMILES: *N1CCCCC1] | 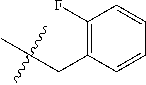 [SMILES: *Cc1ccccc1F] | CH₂CH₃ | H | O |
| 37 | 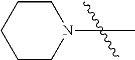 [SMILES: *N1CCCCC1] | 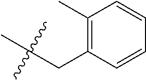 [SMILES: *Cc1ccccc1C] | CH₂CH₃ | H | O |
| 38 | 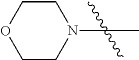 [SMILES: *N1CCOCC1] | Phenyl | CH₃ | H | O |
| 39 | 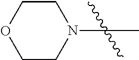 [SMILES: *N1CCOCC1] | 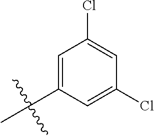 [SMILES: *c1cc(Cl)cc(Cl)c1] | CH₃ | H | O |
| 40 | 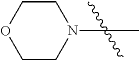 [SMILES: *N1CCOCC1] | 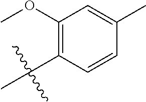 | CH₃ | H | O |
| 41 | 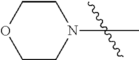 [SMILES: *N1CCOCC1] | 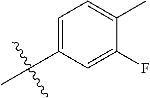 [SMILES: *c1ccc(C)c(F)c1] | CH₃ | H | O |
| 42 | 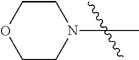 [SMILES: *N1CCOCC1] | 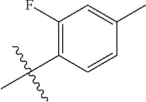 [SMILES: *c1ccc(C)cc1F] | CH₃ | H | O |
| 43 | 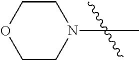 [SMILES: *N1CCOCC1] | 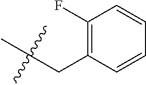 [SMILES: *Cc1ccccc1F] | CH₃ | H | O |
| 44 | 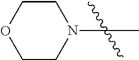 [SMILES: *N1CCOCC1] | 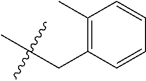 [SMILES: *Cc1ccccc1C] | CH₃ | H | O |
| 45 | 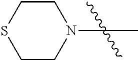 [SMILES: *N1CCSCC1] | Phenyl | CH₃ | H | O |
| 46 | 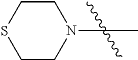 [SMILES: *N1CCSCC1] | 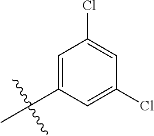 [SMILES: *c1cc(Cl)cc(Cl)c1] | CH₃ | H | O |
| 47 | 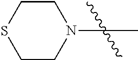 [SMILES: *N1CCSCC1] | 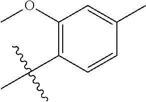 | CH₃ | H | O |
| 48 | 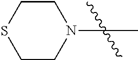 [SMILES: *N1CCSCC1] | 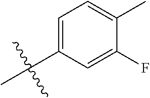 [SMILES: *c1ccc(C)c(F)c1] | CH₃ | H | O |
| 49 | 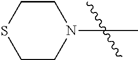 [SMILES: *N1CCSCC1] | 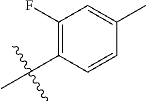 [SMILES: *c1ccc(C)cc1F] | CH₃ | H | O |
| 50 | 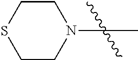 [SMILES: *N1CCSCC1] | 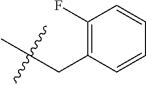 [SMILES: *Cc1ccccc1F] | CH₃ | H | O |
| 51 | 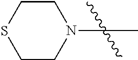 [SMILES: *N1CCSCC1] | 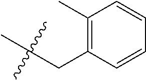 [SMILES: *Cc1ccccc1C] | CH₃ | H | O |
| 52 | 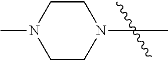 [SMILES: *N1CCN(C)CC1] | Phenyl | CH₃ | H | O |
| 53 | 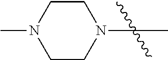 [SMILES: *N1CCN(C)CC1] | 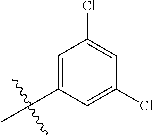 [SMILES: *c1cc(Cl)cc(Cl)c1] | CH₃ | H | O |
| 54 | 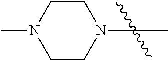 [SMILES: *N1CCN(C)CC1] | 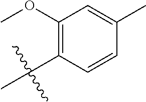 | CH₃ | H | O |
| 55 | 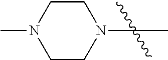 [SMILES: *N1CCN(C)CC1] | 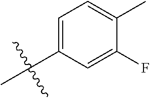 [SMILES: *c1ccc(C)c(F)c1] | CH₃ | H | O |
| 56 | 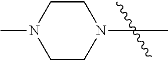 [SMILES: *N1CCN(C)CC1] | 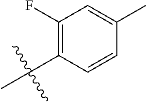 [SMILES: *c1ccc(C)cc1F] | CH₃ | H | O |
| 57 | 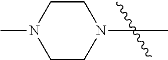 [SMILES: *N1CCN(C)CC1] | 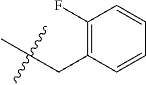 [SMILES: *Cc1ccccc1F] | CH₃ | H | O |
| 58 | 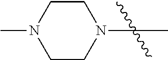 [SMILES: *N1CCN(C)CC1] | 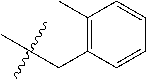 [SMILES: *Cc1ccccc1C] | CH₃ | H | O |
| 59 | 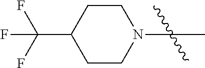 [SMILES: *N1CCC(C(F)(F)F)CC1] | Phenyl | CH₃ | H | S |
| 60 | 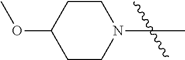 [SMILES: *N1CCC(OC)CC1] | Phenyl | CH₃ | H | S |
| 61 | 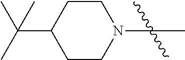 [SMILES: *N1CCC(C(C)(C)C)CC1] | Phenyl | CH₃ | H | S |
| 62 | 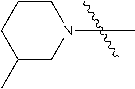 [SMILES: *N1CCCC(C)C1] | Phenyl | CH₃ | H | S |
| 63 | 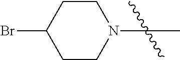 [SMILES: *N1CCC(Br)CC1] | Phenyl | CH₃ | H | S |
| 64 | 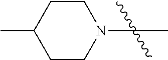 [SMILES: *N1CCC(C)CC1] | 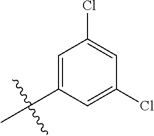 [SMILES: *c1cc(Cl)cc(Cl)c1] | CH₃ | H | S |
| 65 | 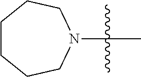 [SMILES: *N1CCCCCC1] | Phenyl | CH₃ | H | S |
| 66 | 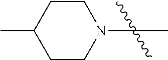 [SMILES: *N1CCC(C)CC1] | Phenyl | CH₃ | H | S |
| 67 | 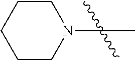 [SMILES: *N1CCCCC1] | 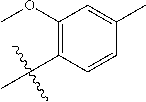 | CH₃ | H | S |
| 68 | 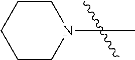 [SMILES: *N1CCCCC1] | 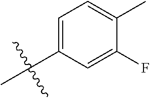 [SMILES: *c1ccc(C)c(F)c1] | CH₃ | H | S |
| 69 | 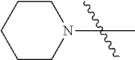 [SMILES: *N1CCCCC1] | 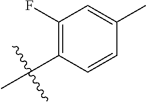 [SMILES: *c1ccc(C)cc1F] | CH₃ | H | S |
| 70 | 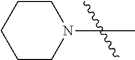 [SMILES: *N1CCCCC1] | 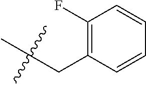 [SMILES: *Cc1ccccc1F] | CH₃ | H | S |
| 71 | 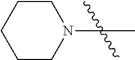 [SMILES: *N1CCCCC1] | 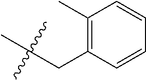 [SMILES: *Cc1ccccc1C] | CH₃ | H | S |
| 72 | 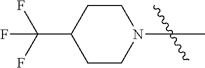 [SMILES: *N1CCC(C(F)(F)F)CC1] | Phenyl | CH₃ | H | NH |
| 73 | 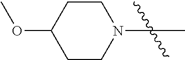 [SMILES: *N1CCC(OC)CC1] | Phenyl | CH₃ | H | NH |
| 74 | 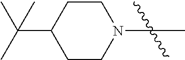 [SMILES: *N1CCC(C(C)(C)C)CC1] | Phenyl | CH₃ | H | NH |
| 75 | 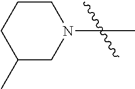 [SMILES: *N1CCCC(C)C1] | Phenyl | CH₃ | H | NH |
| 76 | 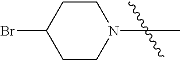 [SMILES: *N1CCC(Br)CC1] | Phenyl | CH₃ | H | NH |
| 77 | 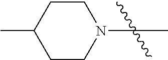 [SMILES: *N1CCC(C)CC1] | 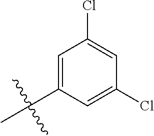 [SMILES: *c1cc(Cl)cc(Cl)c1] | CH₃ | H | NH |
| 78 | 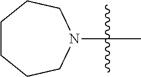 [SMILES: *N1CCCCCC1] | Phenyl | CH₃ | H | NH |
| 79 | 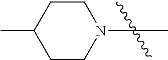 [SMILES: *N1CCC(C)CC1] | Phenyl | CH₃ | H | NH |
| 80 | 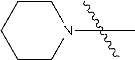 [SMILES: *N1CCCCC1] | 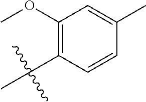 | CH₃ | H | NH |
| 81 | 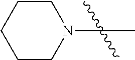 [SMILES: *N1CCCCC1] | 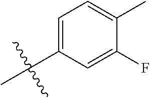 [SMILES: *c1ccc(C)c(F)c1] | CH₃ | H | NH |
| 82 | 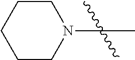 [SMILES: *N1CCCCC1] | 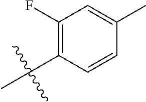 [SMILES: *c1ccc(C)cc1F] | CH₃ | H | NH |
| 83 | 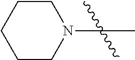 [SMILES: *N1CCCCC1] | 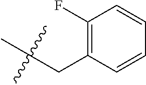 [SMILES: *Cc1ccccc1F] | CH₃ | H | NH |
| 84 | 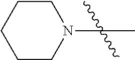 [SMILES: *N1CCCCC1] | 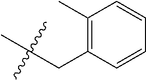 [SMILES: *Cc1ccccc1C] | CH₃ | H | NH |

Exemplary embodiments of the present invention include a compound of Formula (II) or a pharmaceutically acceptable salt form thereof:

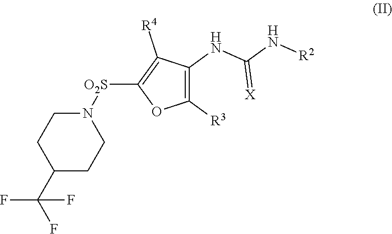

wherein non-limiting examples of R², R³, R⁴, and X are defined herein below in Table 2.

TABLE 2

| Entry | R² | R³ | R⁴ | X |
|---|---|---|---|---|
| 85 | 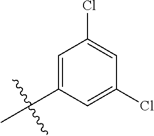 [SMILES: *c1cc(Cl)cc(Cl)c1] | CH₃ | H | O |
| 86 | 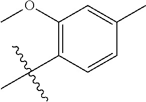 | CH₃ | H | O |
| 87 | 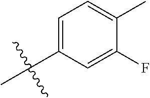 [SMILES: *c1ccc(C)c(F)c1] | CH₃ | H | O |
| 88 | 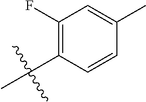 [SMILES: *c1ccc(C)cc1F] | CH₃ | H | O |
| 89 | 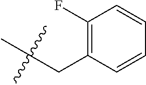 [SMILES: *Cc1ccccc1F] | CH₃ | H | O |
| 90 | 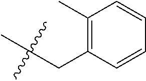 [SMILES: *Cc1ccccc1C] | CH₃ | H | O |
| 91 | 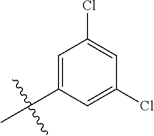 [SMILES: *c1cc(Cl)cc(Cl)c1] | CH₃ | H | S |
| 92 | 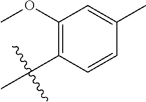 | CH₃ | H | S |
| 93 | 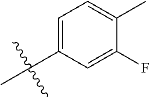 [SMILES: *c1ccc(C)c(F)c1] | CH₃ | H | S |
| 94 | 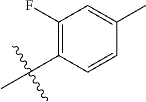 [SMILES: *c1ccc(C)cc1F] | CH₃ | H | S |
| 95 | 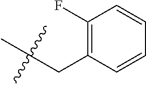 [SMILES: *Cc1ccccc1F] | CH₃ | H | S |
| 96 | 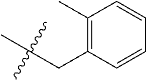 [SMILES: *Cc1ccccc1C] | CH₃ | H | S |
| 97 | 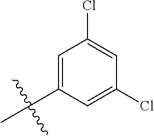 [SMILES: *c1cc(Cl)cc(Cl)c1] | CH₃ | H | NH |
| 98 | 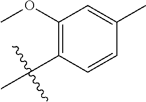 | CH₃ | H | NH |
| 99 | 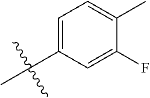 [SMILES: *c1ccc(C)c(F)c1] | CH₃ | H | NH |
| 100 | 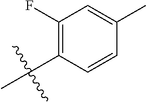 [SMILES: *c1ccc(C)cc1F] | CH₃ | H | NH |
| 101 | 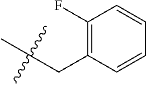 [SMILES: *Cc1ccccc1F] | CH₃ | H | NH |
| 102 | 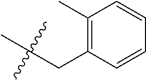 [SMILES: *Cc1ccccc1C] | CH₃ | H | NH |

For the purposes of demonstrating the manner in which the compounds of the present invention are named and referred to herein, the compound having the formula:

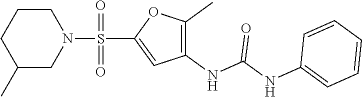 [SMILES: Cc1oc(S(=O)(=O)N2CCCC(C)C2)cc1NC(=O)Nc1ccccc1]

has the chemical name 1-(2-methyl-5-((3-methylpiperidin-1-yl)sulfonyl)furan-3-yl)-3-phenylurea.
For the purposes of the present invention, a compound depicted by the racemic formula, for example:

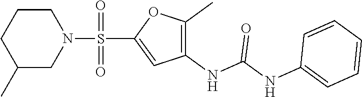 [SMILES: Cc1oc(S(=O)(=O)N2CCCC(C)C2)cc1NC(=O)Nc1ccccc1]

will stand equally well for either of the two enantiomers having the formula:

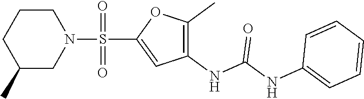 [SMILES: Cc1oc(S(=O)(=O)N2CCC[C@H](C)C2)cc1NC(=O)Nc1ccccc1]

or the formula:

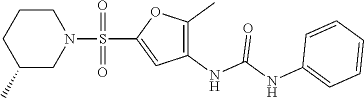 [SMILES: Cc1oc(S(=O)(=O)N2CCC[C@@H](C)C2)cc1NC(=O)Nc1ccccc1]

or mixtures thereof, or in the case where a second chiral center is present, all diastereomers.

In all of the embodiments provided herein, examples of suitable optional substituents are not intended to limit the scope of the claimed invention. The compounds of the invention may contain any of the substituents, or combinations of substituents, provided herein.

### Method of Preparation

The present invention further relates to a process for preparing the endothelial lipase inhibitors of the present invention.

Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatograpy (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of these teachings can be prepared by methods known in the art of organic chemistry. The reagents used in the preparation of the compounds of these teachings can be either commercially obtained or can be prepared by standard procedures described in the literature. For example, compounds of the present invention can be prepared according to the method illustrated in the General Synthetic Schemes:

The reagents used in the preparation of the compounds of this invention can be either commercially obtained or can be prepared by standard procedures described in the literature. In accordance with this invention, compounds in the genus may be produced by one of the following reaction schemes.

Compounds of formula (I) may be prepared according to the process outlined in Schemes 1-3.

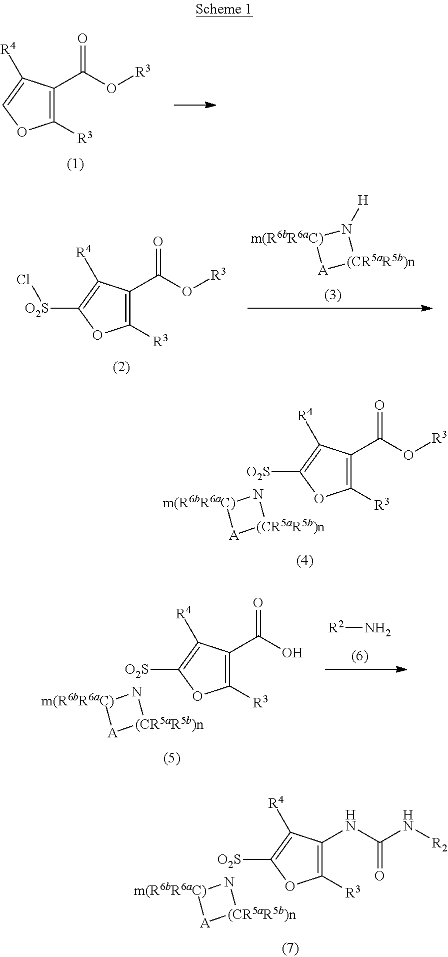

A compound of the formula (1), a known compound or a compound prepared by known methods, is reacted with chlorosulfonic acid, in the presence of phosphorous pentachloride, in an organic solvent such as methylene chloride, dichloroethane, 1,4-dioxane, tetrahydrofuran, 1,2-dimethoxyethane, N,N-dimethylformamide, and the like, optionally in the presence of a base such as pyridine, 2,6-lutidine, triethyl amine, diisopropylethylamine, and the like, optionally with heating, optionally with microwave irradiation to provide a compound of the formula (2). A compound of the formula (2) is then reacted with a compound of the formula (3), a known compound or a compounds prepared by known methods, in an organic solvent such as methylene chloride, dichloroethane, 1,4-dioxane, tetrahydrofuran, 1,2-dimethoxyethane, N,N-dimethylformamide and the like, optionally in the presence of a base such as pyridine, 2,6-lutidine, triethyl amine, diisopropylethylamine, and the like, optionally with heating, optionally with microwave irradiation to provide a compound of the formula (4). A compound of the formula (4) is then reacted with a base such as sodium hydroxide, lithium hydroxide, potassium hydroxide, and the like in an organic solvent such as methanol, ethanol, isopropanol, N,N-dimethylformamide, and the like, optionally with heating, optionally with microwave irradiation to provide a compound of the formula (5). A compound of the formula (5) is then reacted with diphenylphosphoryl azide, optionally in the presence of a base such as pyridine, 2,6-lutidine, triethyl amine, diisopropylethylamine, and the like, in an organic solvent such as methylene chloride, dichloroethane, 1,4-dioxane, tetrahydrofuran, toluene, 1,2-dimethoxyethane, N,N-dimethylformamide, and the like, in the presence of a compound of the formula (6), a known compound or a compound prepared by known methods, optionally in the presence of an coupling agent such as O-(benzotriazol-1-yl)-N,N,N′,N′-tetramethyluronium hexafluorophosphate, N,N′-dicyclohexylcarbodiimide, 1-ethyl-3-(3-dimethylaminopropyl) carbodiimide, and carbonyl diimidazole, optionally with heating, optionally with microwave irradiation to provide a compound of the formula (7).

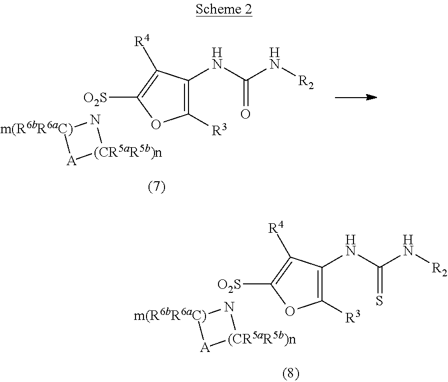

A compound of the formula (7) is reacted with Lawesson reagent (2,4-bis(4-methoxyphenyl)-1,3,2,4-dithiadiphosphetane-2,4-disulfide) in an organic solvent such as methylene chloride, dichloroethane, 1,4-dioxane, tetrahydrofuran, toluene, 1,2-dimethoxyethane, dimethylsulfoxide and the like, optionally with heating, optionally with microwave irradiation to provide a compound of the formula (8). Alternatively, a compound of the formula (7) is reacted with phosphorous pentasulfide in an organic solvent such as methylene chloride, dichloroethane, 1,4-dioxane, tetrahydrofuran, toluene, 1,2-dimethoxyethane, dimethylsulfoxide and the like, optionally with heating, optionally with microwave irradiation to provide a compound of the formula (8).

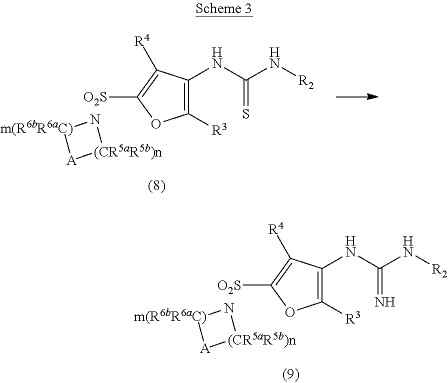

A compound of the formula (8) is reacted bromoethane in an organic solvent such as methanol, ethanol, 1,4-dioxane, tetrahydrofuran, 1,2-dimethoxyethane and the like, optionally with heating, optionally with microwave irradiation. The resulting material is then reacted with ammonia in an organic solvent such as methanol, ethanol, 1,4-dioxane, tetrahydrofuran, 1,2-dimethoxyethane and the like, optionally with heating, optionally with microwave irradiation to provide a compound of the formula (9).

### Formulations

The present invention also relates to compositions or formulations which comprise the endothelial lipase inhibitors according to the present invention. In general, the compositions of the present invention comprise an effective amount of one or more functionalized furan-2-sulfonamides and salts thereof according to the present invention, which are effective for inhibiting endothelial lipase; and one or more excipients.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present invention have improved cellular potency, pharmacokinetic properties, as well as improved oral bioavailability.

The present teachings also provide pharmaceutical compositions that include at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present invention can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known coronary artery disease therapies. Oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present invention intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present invention, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In one embodiment, the form has a viscosity that allows it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

Compounds of the present teachings can be useful for the treatment or inhibition of a pathological condition or disorder in a mammal, for example, a human subject. The present teachings accordingly provide methods of treating or inhibiting a pathological condition or disorder by providing to a mammal a compound of the present teachings including its pharmaceutically acceptable salt) or a pharmaceutical composition that includes one or more compounds of the present teachings in combination or association with pharmaceutically acceptable carriers. Compounds of the present teachings can be administered alone or in combination with other therapeutically effective compounds or therapies for the treatment or inhibition of the pathological condition or disorder.

Non-limiting examples of compositions according to the present invention include from about 0.001 mg to about 1000 mg of one or more functionalized furan-2-sulfonamides according to the present invention and one or more excipients; from about 0.01 mg to about 100 mg of one or more functionalized furan-2-sulfonamides according to the present invention and one or more excipients; and from about 0.1 mg to about 10 mg of one or more functionalized furan-2-sulfonamides according to the present invention; and one or more excipients.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compositions of the present invention and practice the claimed methods. The following working examples therefore, specifically point out the preferred embodiments of the present invention, and are not to be construed as limiting in any way the remainder of the disclosure.

### Example 1

### Evaluation and Selection of Compounds as Endothelial Lipase Inhibitors

Endothelial Lipase Isolated Enzyme Assay: To assay for Endothelial Lipase activity, 15 μl of assay buffer (HBSS without calcium, magnesium, or phenol red, with 25 mM HEPES) was placed in a 384-well plate. Three microliters of PLAT substrate (50 μM, (PED-A1, (N-((6-(2,4-DNP)Amino)Hexanoyl)-1-(BODIPY® FL C5)-2-Hexyl-Sn-Glycero-3-Phosphoethanolamine), Life Technologies catalog # A10070)) dissolved in DMSO was added for a final substrate concentration of 5 μM. The plate was incubated for 10 min at 37° C. to avoid the lag phase. Endothelial Lipase (12 μl; for a final concentration of 0.4 μM) was added for a final assay volume of 30 μl. Fluorescence signal was monitored for 40 min at 37° C. with a plate reader in kinetic mode (80 cycles; kinetic interval, 30 s) with an excitation wavelength of 490 nm and an emission wavelength of 515 nm. Linear regression of the fluorescence intensity values collected from 400 to 1,500 s was used to calculate the reaction rate (the slope), and slopes were used to calculate IC50 values where appropriate. The amount of BODIPY-labeled product generated was calculated at the 30 minute time point as determined from standard curve analysis of purified BODIPY FL C5.

Endothelial Lipase Cellular Assay: To assay for cell surface lipase activity, cells expressing human endothelial lipase (EL) were plated in 384-well plates in 25 μL serum free medium at a density of 2000 cells/well. After 18-24 hours incubation at 37° C., the medium was removed and replaced with 15 μL assay buffer [Hank's Buffered Saline Solution with 25 mM HEPES pH 7.2] and 15 μL PLAT substrate (PED-A1, (N-((6-(2,4-DNP)Amino)Hexanoyl)-1-(BODIPY® FL C5)-2-Hexyl-Sn-Glycero-3-Phosphoethanolamine), Life Technologies catalog # A10070) for a final concentration of 10 μM. Fluorescence signal was monitored for 30 minutes at 37° C. on a plate reader in kinetic mode (60 cycles, kinetic interval: 30 seconds) with an excitation wavelength of 490 nm and an emission wavelength of 515 nm. Linear regression of the fluorescence intensity collected from 480 to 1500 seconds was used to calculate the reaction rate (the slope) and the slopes were used to calculate IC50 values where appropriate. The amount of BODIPY-labeled product generated was calculated at the 30 minute time point as determined from standard curve analysis of purified BODIPY FL C5. In all studies using the inhibitor Ebelactone B, consistent results were obtained when it was dissolved as a stock in DMSO, immediately before use.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound having the formula (I): 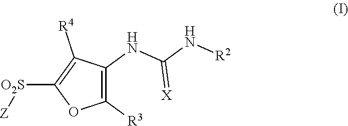
including hydrates, solvates, enantiomers, diastereomers, pharmaceutically acceptable salts, prodrugs and complexes thereof, wherein:
A is selected from a group consisting of CR^{1a}R^{1b}, sulfur, oxygen, and NR⁷;
X is selected from a group consisting of oxygen, sulfur, and NH;
Z is 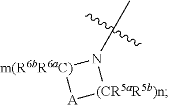
R^{1a} and R^{1b} are each independently selected from a group consisting of optionally substituted C₁-C₆ alkyl, optionally substituted C₃-C₈ cycloalkyl, optionally substituted aryl, optionally substituted arylalkyl, and optionally substituted heteroaryl;
R^{1a} and R^{1b} are taken together with the atoms to which they are bound to form a ring containing 3 to 6 atoms;
R² is selected from a group consisting of optionally substituted C₃-C₇ cycloalkyl, optionally substituted aryl, optionally substituted heteroaryl, optionally substituted biphenyl, 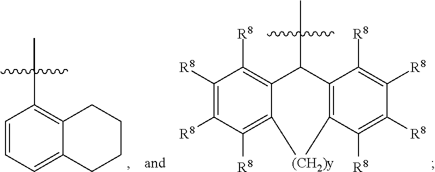
R³ is selected from a group consisting of hydrogen, C₁-C₆ alkyl, and C₃-C₇ cycloalkyl;
R⁴ is selected from a group consisting of hydrogen, C₁-C₆ alkyl, and C₃-C₇ cycloalkyl;
R^{5a} nd R^{5b} are at each occurrence independently selected from a group consisting of hydrogen, optionally substituted C₁-C₆ alkyl, and optionally substituted C₃-C₇ cycloalkyl;
R^{5a} and R^{5b} are taken together with the atoms to which they are bound to form a ring containing 3 to 6 atoms;
R^{6a} nd R^{6b} are at each occurrence independently selected from a group consisting of hydrogen, optionally substituted C₁-C₆ alkyl, and optionally substituted C₃-C₇ cycloalkyl;
R^{6a} and R^{6b} are taken together with the atoms to which they are bound to form a ring containing 3 to 6 atoms;
R^{6a} and R^{6b} are taken together with the atoms to which they are bound to form a ring containing 3 to 6 atoms;
R⁷ is selected from a group consisting of hydrogen, optionally substituted C₁-C₆ alkyl, and optionally substituted C₃-C₇ cycloalkyl;
R⁸ at each occurrence is independently selected from a group consisting of hydrogen, halogen, cyano, optionally substituted C₁-C₆ alkyl, optionally substituted C₁-C₆ alkoxy, optionally substituted C₃-C₈ cycloalkyl, OH, NH₂, NH(C₁-C₆ alkyl), N(C₁-C₆ alkyl)₂, NO₂, C₁₋₃ haloalkyl, C₁₋₃ haloalkoxy, SH, S (C₁-C₆ alkyl), and 3-10 membered cycloheteroalkyl containing 1 to 4 heteroatoms selected from N, O and S;
m=1, 2, 3, or 4;
n=1, 2, 3, or 4;
y=0, 1, or 2;
with the proviso that the compound is not the compound of formula (II):

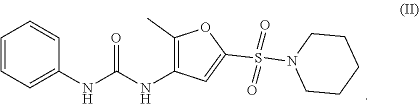 [SMILES: Cc1oc(S(=O)(=O)N2CCCCC2)cc1NC(=O)Nc1ccccc1]

2. The compound of claim 1, having the formula (III): 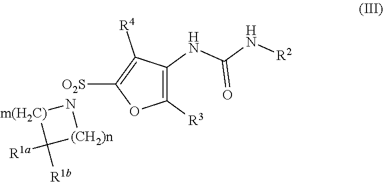
including hydrates, solvates, pharmaceutically acceptable salts, prodrugs and complexes thereof.

3. The compound of claim 1, having the formula (IV): 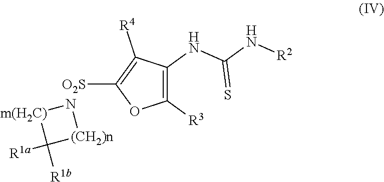
including hydrates, solvates, pharmaceutically acceptable salts, prodrugs and complexes thereof.

4. The compound of claim 1, having the formula (V): 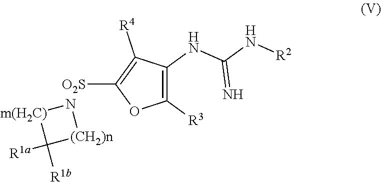
including hydrates, solvates, pharmaceutically acceptable salts, prodrugs and complexes thereof.

5. The compound of claim 1, having the formula (VI): 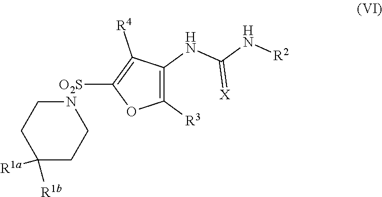
including hydrates, solvates, pharmaceutically acceptable salts, prodrugs and complexes thereof.

6. The compound of claim 1, having the formula (VII): 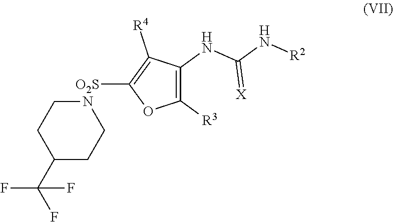
including hydrates, solvates, pharmaceutically acceptable salts, prodrugs and complexes thereof.

7. A composition comprising an effective amount of at least one compound according to claim 1 and at least one pharmaceutically acceptable excipient.

8. A composition comprising an effective amount of at least one compound according to claim 2 and at least one pharmaceutically acceptable excipient.

9. A composition comprising an effective amount of at least one compound according to claim 3 and at least one pharmaceutically acceptable excipient.

10. A composition comprising an effective amount of at least one compound according to claim 4 and at least one pharmaceutically acceptable excipient.

11. A composition comprising an effective amount of at least one compound according to claim 5 and at least one pharmaceutically acceptable excipient.

12. A composition comprising an effective amount of at least one compound according to claim 6 and at least one pharmaceutically acceptable excipient.

13. A method of decreasing endothelial lipase activity in a subject, said method comprising administering to a subject an effective amount of at least one compound according to claim 1.

14. The method of claim 13, wherein the at least one compound is administered in a composition further comprising at least one excipient.

15. A method of treating a disease associated with aberrant endothelial lipase activity in a subject, said method comprising administering to a subject an effective amount of at least one compound according to claim 1.

16. The method of claim 15, wherein the at least one compound is administered in a composition further comprising at least one excipient.

17. A method of increasing high density lipoprotein-cholesterol (HDL-C) in a subject, said method comprising administering to a subject an effective amount of at least one compound according to claim 1.

18. The method of claim 17, wherein the at least one compound is administered in a composition further comprising at least one excipient.

19. A method of treating coronary artery disease in a subject, said method comprising administering to a subject an effective amount of at least one compound according to claim 1.

20. The method of claim 20, wherein the at least one compound is administered in a composition further comprising at least one excipient.

21. A compound selected from the group consisting of

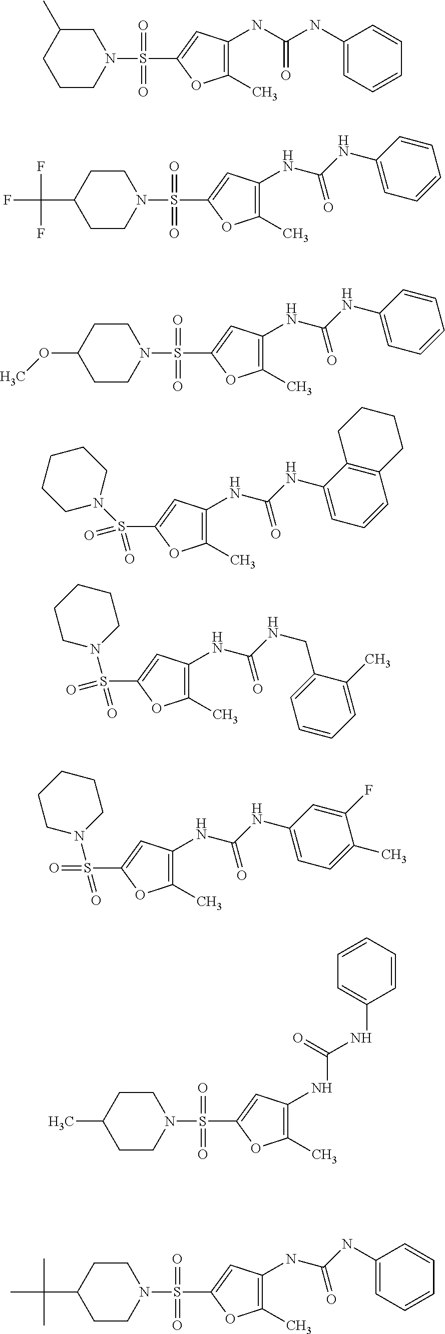

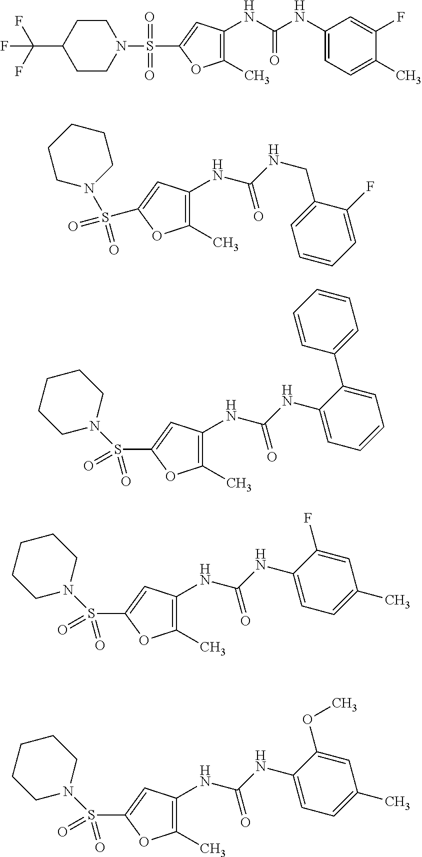

22. A composition comprising an effective amount of at least one compound according to claim 1 and at least one pharmaceutically acceptable excipient.
